(19) United States
(12) Patent Application Publication
(10) Pub. No.: US 2017/0127971 A1
(43) Pub. Date: May 11, 2017

(54) METHOD AND SYSTEM FOR MEASURING PHYSIOLOGICAL PARAMETERS OF A SUBJECT UNDERGOING AN OLFACTORY STMULATION

(71) Applicants: Consiglio Nazionale delle Ricerche, Roma (IT); Universita del Salento, Lecce (IT)

(72) Inventors: Sara Invitto, Lecce (IT); Giovanni Montagna, Roma (IT); Simonetta Capone, Roma (IT); Pietro Siciliano, Roma (IT)

(21) Appl. No.: 15/412,265

(22) Filed: Jan. 23, 2017

Related U.S. Application Data
(63) Continuation-in-part of application No. PCT/IB2015/055575, filed on Jul. 23, 2015.

(30) Foreign Application Priority Data
Jul. 23, 2014 (IT) MI2014A001344
Jul. 23, 2015 (IB) PCT/IB2015/055575

(51) Int. Cl. A61B 5/0484; A61B 5/00

(52) U.S. Cl. CPC A61B 5/04847; A61B 5/7246; A61B 5/743

(57) ABSTRACT

Method and related system for measuring physiological parameters of a human subject undergoing an olfactory stimulation comprising one or more smelling olfactory stimuli, where by means of said system and according to said method a human subject is undergone to said olfactory stimulation by sending said one or more smelling olfactory stimuli to the human subject; one or more physiological parameters of the human subject are recorded; and said olfactory stimulation and physiological parameters recording are synchronized.

### RELATED APPLICATION DATA

This application is a continuation-in-part of International Patent Application No. PCT/IB2015/055575 filed Jul. 23, 2015, which claims priority of Italian Patent Application No. MI2014A001344, filed Jul. 23, 2014, all of which are hereby incorporated by reference in their entireties.

### FIELD OF APPLICATION

The present invention relates to clinical examinations. More specifically, the present invention relates to clinical examinations regarding the recording and graphic representation of the activities of a specific internal organ, especially the brain.

Specifically, the present invention relates to a method and related system for measuring event-related potentials (ERPs) of the brain. In particular, by means of an electroencephalography (EEG) under olfactory stimulation.

### PRIOR ART

In the context of clinical studies, several systems are known for detecting bio-signals, i.e. those chemical, electrical, mechanical or magnetic signals, observable with different degrees and in different manner in living beings and usable to obtain useful information on clinical processes.

In particular, systems for measuring event-related potentials (ERPs) of the brain are known.

Sensory Evoked Potentials (SEPs) are generally electroencephalographic signals being generated when a person responds to a sensory stimulus. SEPs can be of different kind, such as, for example, evoked potentials in response to stimulation of sight, touch or sound.

The brainwaves activity of a human subject can be monitored by means of the electroencephalographic signals perceived on the skull thereof, essentially placing electrodes on the head of the human subject so as to detect his/her EEG activity. During the reading, the electroencephalographic apparatus provides a real time graphic representation of the recorded pattern, which is called electroencephalogram. Such recorded pattern is shown on thermal paper or graph paper, or on monitors with recording on hard disk, CD or DVD. In particular, said record pattern shows the time on the horizontal axis, while the intensity of the perceived potential is shown on the vertical axis.

In particular, nowadays the analysis and the subsequent evaluation of the responses in terms of EEG of a human subject undergoing olfactory stimuli are considered particularly useful, because the brain response to an olfactory stimulus seems to be indicative of the human subject condition, not only from a purely clinical point of view, but also from a behavioral one, with consequent psychological implications.

For example, a particularly advantageous use may be the diagnostic one; in fact, recent studies claim that there is a correlation between the responses to olfactory stimuli and the occurrence of specific degenerative diseases. If the hypotheses of said studies were correct, then it would be possible to use those olfactory stimulations as a diagnostic method.

A further use could be the one related to the therapeutic context. In particular, it is possible that a strong smelling stimulation can act as a stimulus for the revival of an otherwise still nervous activity; or it will be also possible to investigate how the perception of a pleasant substance can have a calming effect on a given human subject.

However, thus far, the study of olfactory ERPs has been conducted by means of simple experiments of olfactory stimulation without any synchronization between the real occurrence of the olfactory stimulus and the collection of the brain information, resulting in a limitation of the reliability and interpretability of the data. On the other hand, a reliable synchronization would be appropriate, especially considering the fact that the emotional response of a given human subject to an olfactory stimulus is independent of his/her will (i.e. it is autonomous) and that the subject under examination has awareness of own answer only a full second after the brain has elaborated such a stimulus.

The use of tubes, for example built with Teflon, is provided in some of the currently used methods for studying the brain potentials related to an olfactory stimulus, the tubes being used in order to carry the smell under examination to a nozzle connected to a funnel-shaped structure positioned 12-15 cm away from the nostrils of the human subject.

However, in this kind of systems, the measurement of the brain potentials is not accurate; in particular, the brain activity of the human subject is conditioned by a variety of visual and auditory factors and, accordingly, is not representative of the administered olfactory stimulus. Therefore, there is the need to visually and acoustically isolate the human subject in order to prevent, or at least minimize, external conditioning of a different nature from the olfactory one.

In other currently known systems, manual methods of administration of the smelling stimuli are used. The administration of the smelling stimuli takes place by means of repeated passages of a series of opaque vials containing different smells in front of the human subject's nose. In particular, these vials are positioned for about 2 seconds 1-2 cm away from the nostrils of the human subject. In order to have a sufficient number of tests with a given sample, those administrations are repeated from 20 to 30 times with a 4-seconds time-distance from each other.

However, even in this way, the measurement of the brainwaves is not very indicative of the actual brain response to the olfactory stimulus: in fact, the olfactory stimulus, which is manually administered, does not allow an accurate correlation between the actual perception of the olfactory stimulus and the corresponding response.

Finally, other prior art methods employ air dilution olfactometers able to carry smelling stimuli with a steady air flow; those systems also propose a first computerized control for sending the olfactory stimuli to the human subject, but do not comprise any synchronization with the electroencephalographic apparatus.

Hence, the total absence of a synchronization leads to unreliable EEG readings. In particular, there is not a signal on the EEG pattern marking the start of the administration of the olfactory stimulus, all the above with a consequent alteration of the analysis and interpretation of the data. This involves the fact that it is not possible subsequently to build an averaging on the pattern and to highlight an evoked potential or an event being directly related. The peculiarity of the apparatus is to mark on the EEG pattern the triggers of the olfactory stimulation. Recent systems do not have a procedure that allows visualizing and measuring whether a change in electroencephalographic signal is induced or modulated by an olfactory stimulus.

Moreover, in the above-described methods according to the prior art, there is always a de-synchronization in the awake stage (absence of olfactory stimulation or exposure to neutral olfactory stimulation) and a stimulation in the EEG caused by both external events linked to sight and hearing and to the normal operation of the brain. Such a de-synchronization ensures the evoked potential or related event to be visible after numerous and repeated stimulations.

Hence, the need not only to repeat multiple stimulations to be able to observe the actual brain response to the olfactory stimulus, but also to achieve an average of the signals related to olfactory stimuli of the same duration in order to adequately compensate for the background noise, and then making the read signal as much relevant as possible to the sole activity of olfactory stimulation.

On the basis of the aforementioned considerations, the present invention relates to a method and related system allowing to overcome (or at least minimize) the typical drawbacks that affect the methods and related systems of the prior art. In particular, the purpose of the present invention is to propose a method and related system allowing the display of a marker being produced by the trigger of the olfactory stimulation by means of an amplifier interfacing the trigger with the electroencephalographic recording. Moreover, it will be thus possible to standardize the methodology of virtual olfactory stimulation to known visual or auditory stimulation methodologies, for which the synchronization of the sensory stimulation events is implemented by specific stimuli presentations software.

### SUMMARY OF THE INVENTION

The present invention is based on the general consideration that the disadvantages and/or limitations of the methods and systems according to the prior art can be overcome or at least partially solved by means of a system wherein the sending of the olfactory stimuli to the human subject is interfaced by means of a marking on the acquisition system of the electroencephalographic signal. Subsequently, this allows a direct averaging processing on the pattern to assess changes induced and/or modulated by the olfactory stimulus administered in a temporal range marked on the pattern and with a stimulation intensity measurable and changeable by the experimenter.

To this end, the present invention is based on the further consideration that by digitizing the operations by means of the use of programmable means automating and regulating the whole procedure of administration of the olfactory stimuli and of acquisition of the encephalogram data, it is possible to obtain the desired synchronization and then even more reliable results. In particular, in a non-limiting preferred embodiment of the present invention, the procedure (measurement) provides the regulation in terms of intensity and duration of the different olfactory stimuli which the human subject is subjected to.

On the basis of the aforementioned considerations, the present invention has as its first object a method for the brain parameters recording of a human subject undergoing an olfactory stimulation comprising one or more smelling olfactory stimuli, said method comprising the steps of:
Submit the human subject to such olfactory stimulation by sending said one or more smelling olfactory stimuli to the human subject;
Generation and Recording of an electroencephalogram of the human subject;
   said method further comprising the generation of a trigger signal and in that the signal is used by the EEG apparatus to affix one or more markers to the EEG pattern.

In another non-limiting preferred embodiment of the present invention, said method comprises the measurement of the brain potentials (voltage variations) of said human subject.

Advantageously, said trigger signal can be generated at the start time of said olfactory stimulation, and at least one label (marker) can be affixed to the EEG pattern to mark the start time of the olfactory stimulation.

Advantageously, said olfactory stimulation may comprise one or more neutral olfactory stimuli, where said smelling olfactory stimuli may be obtained, for example, by using one or more corresponding smelling substances.

A further object of the present invention is a system for measuring the brain parameters of a human subject undergone an olfactory stimulation according to a method as briefly described above, said system comprising:

means adapted to generate and send to a human subject said olfactory stimulation comprising said one or more smelling olfactory stimuli;
electronic means for the operational management of said means adapted to generate and send said one or more smelling olfactory stimuli;
measurement means adapted to record an electroencephalographic pattern;
wherein said programmable means are interfaced with said electronic means and adapted to set an olfactory stimulation, wherein said electronic means are adapted to generate and forward to said measurement means a trigger signal, and wherein said measurement means are configured so as to affix at least one marker when receiving said trigger signal to said electroencephalographic pattern.

For example, said electronic means may be configured so as to generate and forward to said measurement means said trigger signal at the start time of said olfactory stimulation, and said measurement means can be configured so as to affix at least one marker to mark the start time of said olfactory stimulation to said electroencephalographic pattern.

Advantageously, said means adapted to generate and send to a human subject said one or more smelling olfactory stimuli may comprise:

One or more containers (106, 108) for one or more smelling substances (109);

Means (112) adapted to enrich a first main air flow of said one or more smelling substances and to send to said human subject said enriched air flow.

Further non-limiting preferred embodiment of the method and system according to the present invention are defined in the dependent claims.

Additional features and advantages of the method and related system according to the present invention for measuring physiological parameters of a human subject undergoing an olfactory stimulation are thoroughly discussed in the following description of the non-limiting preferred embodiments shown in the drawings, given for example purpose only.

### BRIEF DESCRIPTION OF THE DRAWINGS

FIG. 1 schematically shows a system for measuring brain parameters of a human subject undergoing an olfactory stimulation according to one non-limiting preferred embodiment of the present invention;

FIG. 2 shows an example of a method for measuring brain parameters of a patient with related administration of a series of smelling and neutral olfactory stimuli according to one non-limiting preferred embodiment of the present invention.

### DETAILED DESCRIPTION OF THE PRESENT INVENTION

Although, in the following, some specific preferred embodiments of the system and method according to the present invention shown in the drawings are described, it should be noted that the present invention is not limited to the embodiments shown in the drawings and described below; on the contrary, the following description explains several aspects of the present invention, while the purpose and scope thereof are defined by the claims.

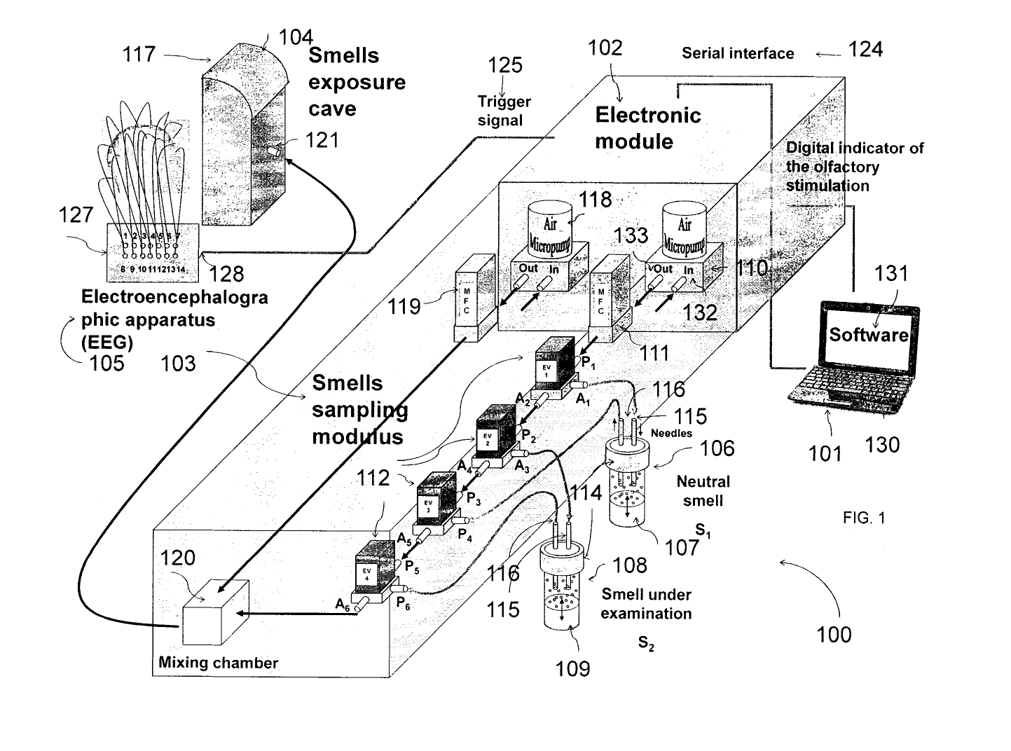

With reference to FIG. 1, a system according to the embodiment of the present invention represented therein for measuring brain parameters of a human subject undergoing an olfactory stimulation is now described. Specifically, this relates to the reading by means of an electroencephalogram of event-related brain potentials.

As shown in FIG. 1, with 100 is globally indicated a system according to the embodiment of the present invention represented therein. The system 100 comprises programmable means 101, electronic means 102, means for sending olfactory stimuli 103 and an EEG apparatus 105. It should be clarified that, in the following, the term “olfactory stimuli” will indiscriminately refer to the smelling olfactory stimuli and to the neutral olfactory stimuli, respectively obtained by means of one or more smelling substances and one or more substances being neutral from the olfactory point of view.

In particular, the means for sending olfactory stimuli 103 are adapted to send, along appropriate paths, the olfactory stimuli to a human subject (not indicated in the figure). Such means for sending olfactory stimuli 103 comprise a first container 106 and a second container 108. Said first container 106 is adapted to contain a reference substance or mixture of substances 107 being neutral from the olfactory point of view (for example distilled water), while inside the second container 108 there is a smelling substance or a mixture of smelling substances under examination 109 (for is example linalool, 3-metilbutilestere, 3-phenylethanol, etc.). The containers 106, 108 are closed on the upper part by closure means 114 adapted to prevent possible leakages of smelling and/or neutral substances contained therein. In the case shown in the figure, the closure means 114 are structurally identical for both containers 106, 108 although different closure means may be provided depending on the needs and/or circumstances.

In the embodiment depicted in FIG. 1, the containers 106 and 108 are in the form of glass vials with a cylindrical body. At the upper end of the cylindrical body, the closure means 114 are in the form of a plug comprising two small pass-wall tubes 115, 116. In particular, an inlet tube (delivery) 115 and an outlet tube (exhaust) 116 of the particular gaseous substances mixture.

The means for sending olfactory stimuli 103 further comprise a first micro-pump 110, a first flow controller 111 and a combination of electro-valves 112 (four in the example shown in the figure, but the number can change depending on the needs and/or circumstances).

With reference to FIG. 1, the first micro-pump 110 generates an air flow by drawing the same from the outside by an inlet tube (delivery) 132 and expelling it through an outlet tube (exhaust) 133. This air flow is then conveyed towards the first flow controller 111, by which it is adjusted and kept constant.

Although not graphically shown in FIG. 1, such first flow controller 111 has input and output means of the air coming from said first micro-pump 110.

The combination of electro-valves 112 comprises in particular four three-way electro-valves. In particular, the electro-valves labeled in FIG. 1 with EV1 and EV2 have one inlet and two outlets of a gas or a gas mixture (for example, air plus a substance being neutral from the olfactory point of view, or air and one or more smelling substances under examination), while the electro-valves labeled with EV3 and EV4 have two inlets and one outlet of that gas or gas mixture. Those inlets and outlets are indicated on each electro-valve with Pᵢ (inlet) and Aᵢ (outlet), respectively, with “i” between 1 and 6. Finally, each electro-valve is connected to one of the two containers to receive or forward the gas or gas mixture.

It will be obvious to a person skilled in the art that, although the system described in the present invention and shown in FIG. 1 has exactly two containers and four electro-valves, this configuration is not intended to limit in any way the scope of the invention itself, since, according to different embodiments, said containers and said electro-valves can arise with a different occurrence compared to that shown in FIG. 1, for example when the purpose is to administer different smelling substances.

In particular, the above also applies for the cascade configuration (in series) of the electro-valves; also in this case it will be clear to a person skilled in the art that said configuration of the electro-valves can be parallelized (entirely or in part), in case, depending on the needs and/or circumstances, and that one or more electro-valves can correspond to each container according to the chosen configuration.

The embodiment of the apparatus according to the present invention shown in FIG. 1 further comprises a second micro-pump 118, a second flow controller 119, and a mixing chamber 120. Said second micro-pump 118 and second flow controller 119 may be structurally and functionally identical to the first micro-pump 110 and to the first flow controller 111, or even different, depending on the needs and/or circumstances. Regarding the second flow controller 119, it is adapted to regulate the amount of air coming out from the second micro-pump 118. The so regulated air flow then reaches the mixing chamber 120, within which occurs the possible dilution of the concentration of the gas mixture containing one or more smelling substances under examination coming from the combination of electro-valves 112. However, to the purposes of the present invention, it will be obvious from the following description that said second micro-pump 118, second flow controller 119 and mixing chamber 120 may be possibly omitted entirely or in part.

Finally, the system shown in FIG. 1 comprises smell exposure means 104.

In the particular embodiment shown in FIG. 1, said smell exposure means 104 are in the form of a Plexiglas cave 117, large enough to house the head of the human subject and suitably darkened to avoid visual conditionings. These smell exposure means 104 also have a pass-wall tube (delivery) 121 adapted to allow the inlet of the air flow containing the olfactory stimulus within the cave 117.

Alternatively, in a further non-limiting preferred embodiment, the Plexiglas cave 117 can be suitably replaced by a simple inhalation mask.

Still with reference to FIG. 1, the electronic means 102 in turn comprise a microcontroller (not shown), an electronic system operating said electro-valves (not shown), a trigger section (not shown) adapted to generate a trigger signal 125 and an interface 124 to send commands from said programmable means 101 to said microcontroller.

It should also be specified that said electronic means 102 are adapted to convert the data sent by the programmable means 101 into electrical pulses for the various pieces of equipment of the system.

In a particularly preferred embodiment of the present invention, the trigger signal 125 being generated by the trigger section is adapted to mark the encephalographic pattern with the purpose of an evaluation and analysis thereof according to latency and amplitude variations of encephalographic components.

To affix more markers to the pattern, more trigger signals are sent according to the olfactory stimulation program set by the management software 131.

Regarding the EEG apparatus 105, the related components of such apparatus belong to the prior art and, therefore, do not need a detailed description.

In the embodiment described in FIG. 1, said EEG apparatus 105 is of commercial nature, has 16 channels and comprises a TTL port 128 for the trigger signal.

In other non-limiting preferred embodiments, the EEG apparatus 105 may present a different range of channels, for example equivalent to 32 or 64 channels, based on the different needs of the operator and the examination in re.

The programmable means 101 are adapted to manage the entire olfactory stimulation process and basically comprise a management software 131 and a hardware 130 on which such management software 130 is executed.

In the present embodiment, the programmable means 101 are in the form of a laptop. However, it will be obvious to those skilled in the art that such programmable means 101 can be represented, in alternative embodiments, by any hardware device able to execute the management software 131.

It will now be described a non-limiting preferred embodiment of a method for measuring physiological parameters (in particular electroencephalographic parameters) of a human subject undergoing an olfactory stimulation, according to the present invention.

In its most general form, such a method comprises the steps of administration of the olfactory stimulation to the human subject and recording of the encephalographic parameters.

In turn, the step of administration of the olfactory stimulation comprises a step of setting the stimulation (i.e. the phase of setting the stimulation program where all the features of a particular stimulation are specified), a step of administration of the olfactory stimulation (i.e. the step of generation and administration of the smelling and/or neutral olfactory stimuli by using the means for sending olfactory stimuli) and a step of data measurement.

It is necessary to specify that the expression “olfactory stimulation” is meant to indicate a sequence of one or more smelling olfactory stimuli possibly different and possibly alternating with one or more neutral olfactory stimuli; the expression “neutral olfactory stimulus” is meant to indicate, for example, a stimulus by means of a gaseous solution of air and evaporated distilled water; and, finally, the expression “smelling olfactory stimulus” indicates, for example, the administration of a gaseous mixture comprising air and a smelling substance.

This step of setting the stimulation is performed by the programmable means 101. In the embodiment of the present invention represented in the figures and described hereafter, an operator will load an olfactory stimulation program by means of the management software 131, where said program will specify:
a total number (No) of smelling olfactory stimuli;
a total number (Nn) of neutral olfactory stimuli;
a total number (N) of smelling and neutral olfactory stimuli (equal to No+Nn) that is the total quantity of smelling and neutral olfactory stimuli presentations that are part of the whole olfactory stimulation process;
the start time of each individual smelling and/or neutral olfactory stimulus;
the presentation time (that is the duration) of each individual smelling olfactory stimulus;
the presentation time (that is the duration) of each individual neutral olfactory stimulus;
the start time of the stimulation, that is the moment when the olfactory stimulation will start;
what the first olfactory stimulus of the olfactory stimulation will be;
a particular presentation sequence or presentation mode, i.e. the sequence or alternation (random or predetermined by the operator) of the smelling and neutral olfactory stimuli of the established presentation mode of the smelling substance under examination 109 compared with the reference smelling substance 107;
the smelling substance of each smelling olfactory stimulus comprised in the olfactory stimulation;
a relative percentage of presentation (Pᵣ), that is the ratio (expressed as a percentage) of the number of smelling olfactory stimuli compared with the neutral olfactory stimuli;
the concentration or dilution ratio of each olfactory stimulus, with which it may be possible to dilute the smelling concentration by means of a different subdivision of the total flow, which is kept constant during the whole sequence of neutral and smelling stimuli, on the flow controllers 111 and 119;
the overall temporal duration of the olfactory stimulation;
a total flow, that is the amount of air at the outlet of each of the two flow controllers 111 and 119;
temporal digital indicators (target) of said olfactory stimulation when the smelling olfactory stimulus comes at the positioning point of the inlet tube of the cave 117;

Specifically, it is found that:
the relative percentage of presentation of the smelling stimulation is given by Pᵣ=(No/(No+Nn))*100;
the olfactory stimulation may be manually set by an operator (manual sequence), or the program may have different stimulations being stored or randomly generated (random sequence);
the overall temporal duration will preferably be equal to at least 15 minutes;
the total flow can be set by the management software 131 (typically 100 ml/min according to the full scale of the flow controllers);
the temporal digital indicators are recorded by the EEG data acquisition software;
the possible dilution ratio allows to change the concentration of said smelling substance under examination 109 while keeping the total flow constant. The maximum concentration of pure smelling substance is given by the substance concentration in the test space of the vial containing the substance, which in turn depends on its saturated vapor pressure at a given pressure and temperature; the minimum concentration of pure smelling substance is instead tied to the minimum flow that can be set on the flow controller 111 based on its accuracy (typically, 1% of the full scale).

When the setting is finished, the microcontroller of the electronic means 102 receives the commands from the programmable means 101 by means of the interface 124, and initiates the olfactory stimulation by acting on the different parts of the system, according to the program.

The trigger section generates said trigger signal 125 synchronously with the programmed stimulation. In particular, the trigger signal 125 allows the EEG to show into the graph of the recorded pattern the start time (i.e. the time when the olfactory stimulation starts). The generation of the trigger signal is repeated for each olfactory stimulation according to the management program 131, so that the generated trigger signals allow to show into graph of the recorded pattern all the start times (i.e. all the times when the olfactory stimulation starts).

In a further non-limiting preferred embodiment, said EEG apparatus 105 may show into the graph of recorded pattern, besides the start and end times of the stimulation, also the nature of stimulation in terms of administered substance, dilution ratio and presentation time.

Once activated by the electronic means 102, the means for sending olfactory stimuli 103 provide for the step of preparation and administration of the olfactory stimulation. As anticipated, by means of the first micro-pump 110, an air flow is generated and conveyed toward the flow controller 111. Downstream the controller/regulator 111, the air flow can follow two different paths, depending on whether the neutral olfactory substance 107 (neutral stimulus) or the smelling substance 109 (smelling stimulus) should be administrated.

If the neutral olfactory substance 107 comprised in the first container 106 should be administered, the electro-valve EV1 is characterized by the closing state of the outlet A₂ and opening state of both the inlet P₁ and outlet A₁. In this way, the air flow is conveyed toward the small inlet tube 115 of the first container 106 and enters the first container 106. Once inside the first container 106, the air flow collects the volatile components of the neutral olfactory substance 107 and then flows out from the first container 106 through the small outlet tube 116. The air flow is then conveyed toward the electro-valve EV3 (in the closing state of the inlet P₃ and opening state of the outlet A₅ and inlet P₄), and then flows into the electro-valve EV4 (whose state is characterized by the closing of the inlet P₆ and by the opening of the outlet A₆ and inlet P₅).

Alternatively, if the smelling substance 109 comprised in the second container 109 should be administered, the electro-valve EV1 state is characterized by the closing of the outlet A₁ and by the opening of the inlet P₁ and outlet A₂. The air flow is then conveyed toward the electro-valve EV2, whose state is characterized by the closing of the outlet A₄ and by the opening of the outlet A₃ and inlet P₂. In this way, the air flow is conveyed toward the small inlet tube 115 of the second container 108 and then into the container, wherein it collects the volatile components of the smelling substance 109 and then flows out from this second container 108 through the small outlet tube 116. At this point, the air flow is conveyed toward the electro-valve EV4 (whose state is characterized by the closing of the inlet P₅ and by the opening of the outlet A₆ and inlet P₆).

Regardless of the followed path, once passed the electro-valve EV4, the air flow is conveyed toward the mixing chamber 120. Inside the mixing chamber 120, the air flow carrying the volatile components of the smelling substance can undergo a dilution process by which it is optionally mixed with the air flow coming from the second flow regulator 119, and so the smelling substance concentration is decreased in percentage terms.

Subsequently, upon the arrival of the air flow into the cave 117, the real phase of administration starts: in fact, the air flow flows out from the mixing chamber 120 and, passing through the pass-wall tube 121, enters the cave 117 to be then inhaled by the human subject therein.

The phase of brain parameters recording is characterized by the recording of the brain electrical activity. The EEG apparatus 105, synchronized by means of the trigger signal 125, affixes a marker on the pattern at the start time of the stimulation. This allows subsequent psycho-physiological analysis derived from the reading and analysis of the pattern.

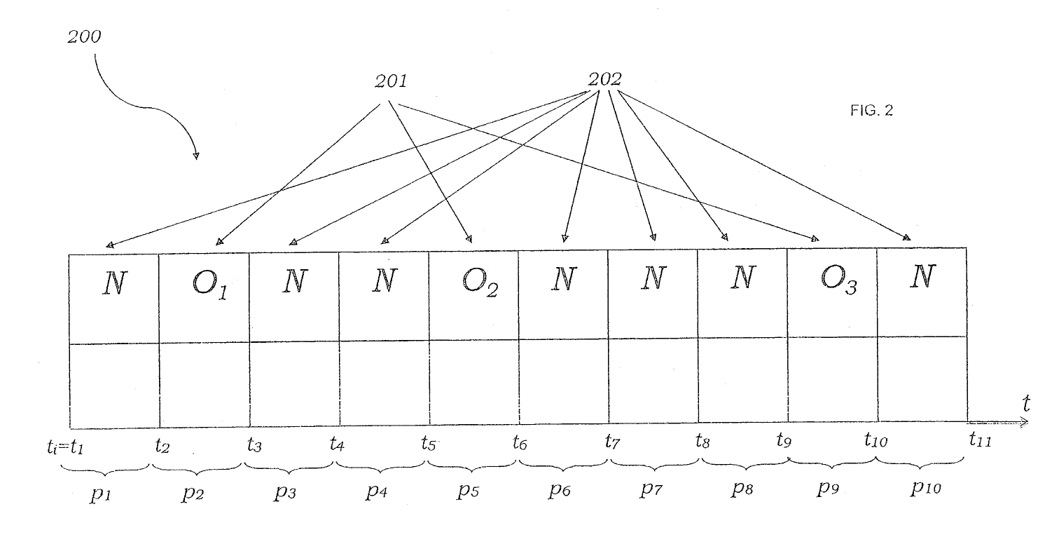

With reference to FIG. 2, the different operations carried out by the system in the case of the olfactory stimulation represented therein will now be described as an example.

In particular, with 200 is globally indicated an olfactory stimulation comprising a plurality of smelling olfactory stimuli 201 and a plurality of neutral olfactory stimuli 202.

Specifically, with regard to the plurality of smelling olfactory stimuli 201, with O₁, O₂, O₃, Oᵢ, are indicated smelling olfactory stimuli deriving from a same smelling substance under examination submitted more than once (i=1, . . . , m) in the same measurement cycle. The response to olfactory stimuli deriving from a different smelling may be recorded by changing the vial containing the smelling substance, and recording another measurement cycle.

As for the plurality of neutral olfactory stimuli 202, with N is indicated a neutral olfactory stimulus deriving from an olfactory neutral reference substance.

On the time axis, with t, t₁, t₂, t₃, . . . t₁₀ are further labeled the start and end times related to the individual olfactory stimuli (smelling and neutral) determining the relative presentation times p₁, p₂, p₃, . . . p₁₀.

Then the operator, by using the programmable means, will set the stimulation program by setting all the features of the olfactory stimulation 200 in the following way, for example:

N=No+Nn=10;
the relative percentage of presentation of the smelling stimulation is given by Pᵣ=(No/(No+Nn))*100=30%; resulting in No=3;
presentation times of the smelling and neutral stimuli (that is the duration): p_{n,o}=1.5 min=90 s; resulting in an overall measurement duration of 15 min;
start time of the olfactory stimulation: tᵢ=0s;
predetermined sequence (manual or random): N, O₁, N, N, O₂, N, N, N, O₃, N;
trigger signal: synchronous ( . . . );
total air flow: 100 ml/min
possible dilution ratio for O₁: 10%;
dilution ratio for O₂: 0%;
dilution ratio for O₃: 40%.

However, typically, in a measurement cycle, the same dilution ratio will be used, so as to repeat more than once the exposure to the olfactory stimulus at a given concentration, as required by the data analysis, while for other dilution ratios a new measurement cycle will be recorded.

After all the parameters have been set, the system initiates the olfactory stimulation 200 at the start time t₁.

The commands are sent from the programmable means to the electronic means, which provide for activating the means for sending smelling and/or neutral olfactory stimuli according to the previously described ways. For example, at the time t₂, the state of the electro-valves will be the following.

EV1: inlet P₁ open; outlet A₁ open; outlet A₂ closed;
EV2: inlet P₂ closed; outlet A₃ closed or open (indifferently); outlet A₄ closed or open (indifferently);
EV3: inlet P₃ open or closed (indifferently); inlet P₄ open; outlet A₅ open;
EV4: inlet P₅ open; inlet P₆ closed; outlet A₆ open.

At the time t₃, the state of the electro-valves will be the following.

EV1: inlet P₁ open; outlet A₁ closed; outlet A₂ open;
EV2: inlet P₂ open; outlet A₃ open; outlet A₄ closed;
EV3: inlet P₃ open or closed (indifferently); inlet P₄ open or closed (indifferently but preferably closed to prevent insertions from the container 107); outlet A₅ open or closed (indifferently but preferably closed);
EV4: inlet P₅ open or closed (indifferently but preferably closed); inlet P₆ open; outlet A₆ open.

Together with the activation of the means for sending olfactory stimuli, the electronic means, by means of the trigger section, further provide for generating a trigger signal that is sent to the EEG apparatus, which marks the EEG pattern upon receiving the trigger signal, in particular it affixes a marker (a label) to the pattern in correspondence of the start time of stimulation.

The administration of said olfactory stimulus will last 90 seconds, as set.

Once the 90 seconds of administration of O₁ have passed, on one hand the electronic means will provide for activating the means for sending olfactory stimuli for the administration of the neutral olfactory stimulus Ni and, on the other hand, according to stimulation paradigm, they will possibly provide for sending a new trigger signal to the EEG apparatus to signal the start of a new olfactory stimulus inside the electroencephalogram. In an analogous way to the stimulus O₁, the means for sending olfactory stimuli will convey the air flow towards the container of the reference smelling substance. The mixture so obtained will not suffer any dilution process inside the mixing chamber (dilution ratio N=0%) and will be conveyed to the exposure means for the overall duration of 90 seconds.

Once the presentation of the second olfactory stimulus has ended, in an analogous way to that shown for the smelling olfactory stimulus O₁ and for the neutral olfactory stimulus N, the system will proceed with the preparation and with the administration of the olfactory stimuli according to the generated sequence N, O₁, N, N, O₂, N, N, N, O₃, N.

At the end of the olfactory stimulation, that is at t₁₁=15 min, the system ends the session.

It has been therefore demonstrated, by means of the detailed description of the embodiment described above, that the present invention allows to achieve the intended purpose and to overcome the typical drawbacks that typically affect the systems and the related methods for reading the physiological parameters under olfactory stimulation according to the prior art.

Although the method and system according to the present invention have been previously clarified by means of the previous detailed description of the embodiments shown in the drawings, the present invention is not limited to the embodiments described above and shown in the drawings.

The purpose of the present invention is therefore defined by the claims.

1. A method recording for the brain parameters of a human subject undergoing an olfactory stimulation comprising one or more smelling olfactory stimuli, the method comprising the steps of:
submitting the human subject to the olfactory stimulation by sending the one or more smelling olfactory stimuli to the human subject;
generating and recording of an electroencephalogram of the human subject; and
generating a trigger signal,
wherein the trigger signal is used by the EEG apparatus to affix one or more markers to the EEG pattern.

2. The method according to claim 1, further comprising the measurement of the brain evoked potentials or voltage variations of the human subject.

3. The method according to claim 1, wherein the trigger signal is generated at the start time of the olfactory stimulation, and wherein at least one marker is affixed to the EEG pattern to mark the start time of the olfactory stimulation.

4. The method according to claim 1, wherein one or more markers are affixed to the EGG pattern based on the olfactory stimulation effectively sent to the human subject.

5. The method according to claim 1, wherein the olfactory stimulation comprises one or more neutral olfactory stimuli.

6. The method according to claim 5, wherein the smelling olfactory stimuli are obtained by using one or more corresponding smelling substances.

7. The method according to claim 1, comprising recording of indications related to the start and/or end time of each smelling and/or neutral olfactory stimulus on the electroencephalogram.

8. The method according to claim 1, wherein the olfactory stimulation has an overall temporal duration equal to at least 15 minutes.

9. A system for measuring brain parameters of a human subject undergoing an olfactory stimulation according to a method as claimed in claim 1, the system comprising:
means adapted to generate and send the olfactory stimulation comprising one or more smelling olfactory stimuli to a human subject;
electronic means for the operational management of the means adapted to generate and send the one or more smelling olfactory stimuli;
measurement means adapted to record an electroencephalographic pattern;
programmable means interfaced with the electronic means and adapted to set an olfactory stimulation
wherein
the electronic means are adapted to generate and forward a trigger signal to the measurement means, and
the measurement means are configured so as to affix at least one marker to the electroencephalographic pattern when receiving the trigger signal.

10. The system according to claim 9, wherein the electronic means are configured so as to generate and forward the trigger signal to the measurement means at the start time of the olfactory stimulation, and wherein the measurement means are configured so as to affix at least one marker to the electroencephalographic pattern to mark the start time of the olfactory stimulation.

11. The system according to claim 9, wherein the means adapted to generate and send the one or more smelling olfactory stimuli to a human subject include:
one or more containers for one or more smelling substances;
means adapted to enrich a first main air flow of the one or more smelling substances and to send the enriched air flow to the human subject.

12. The system according to claim 11, wherein the means adapted to enrich the first main air flow comprise a combination of one or more electro-valves opportunely connected both to each other and to the one and more containers.

13. The system according to claim 11, comprising means adapted to generate the first main air flow and to inlet it in the combination of one or more electro-valves.

14. The system according to claim 12, wherein the one or more electro-valves are connected to each other in series and are connected in parallel with one or more containers.

15. The system according to claim 1, comprising means adapted to generate a second air flow and to mix it with the first main air flow.

16. A method recording for the brain parameters of a human subject undergoing an olfactory stimulation, the method comprising the steps of:
submitting the human subject to the olfactory stimulation by sending one or more smelling olfactory stimuli to the human subject;
generating and recording of an electroencephalogram of the human subject;
generating a trigger signal at the start time of the olfactory stimulation,
wherein the signal is used by the EEG apparatus to affix one or more markers to the EEG pattern, at least one marker being affixed to the EEG pattern to mark the start time of the olfactory stimulation.

17. The method according to claim 16, further comprising the measurement of the brain evoked potentials or voltage variations of the human subject.

18. The method according to claim 16, wherein one or more markers are affixed to the EGG pattern based on the olfactory stimulation effectively sent to the human subject.

19. The method according to claim 16, wherein the olfactory stimulation comprises one or more neutral olfactory stimuli and the smelling olfactory stimuli are obtained by using one or more corresponding smelling substances.

20. The method according to claim 16, comprising recording of indications related to the start and/or end time of each smelling and/or neutral olfactory stimulus on the electroencephalogram.
